(19) United States
(12) Patent Application Publication
(10) Pub. No.: US 2025/0114207 A1
(43) Pub. Date: Apr. 10, 2025

(54) DISC-SHAPED AUGMENTATION FOR A LONG BONE

(71) Applicant: Waldemar Link GmbH & Co. KG, Hamburg (DE)

(72) Inventors: Harm-Iven JENSEN, Noer (DE); Helmut D. LINK, Hamburg (DE)

(73) Assignee: Waldemar Link GmbH & Co. KG, Hamburg (DE)

(21) Appl. No.: 18/684,187

(22) PCT Filed: Aug. 8, 2022
(86) PCT No.: PCT/EP22/72233

§ 371 Date: Dec. 4, 2024

(30) Foreign Application Priority Data
Aug. 16, 2021 (EP) 21000233.3

(51) Int. Cl. A61F 2/38; A61F 2/30

(52) U.S. Cl. CPC A61F 2/389; A61F 2002/30736

(57) ABSTRACT

The invention relates to a disc-shaped augmentation for filling bone defects, in particular at the end of long bones, such as the tibia, comprising a proximal side (12), a distal side (II), an outer casing (15) on lateral sides and an inner wall for a through-opening (2), running from the proximal to the distal side, for a stabilising keel (8) of an endoprosthesis (9) arranged on the proximal side. The augmentation is generally C-shaped with two sections (31, 32) flanking the through-opening. According to the invention, a connection part (30) is formed in an articulated manner between the flanking sections (31, 32) and elastically cooperating with the flanking sections (31, 32).

The invention relates to a disk-shaped augment for filling bone defects, particularly at the end of long tubular bones such as the tibia. The augment is provided with a proximal side, a distal side, an outer sheath on lateral sides, and an inner wall for a through-opening running from the proximal to the distal side for an anchoring keel of an endoprosthesis arranged on the proximal side.

When implanting endoprostheses, in particular joint endoprostheses, a problem sometimes arises in that the bone receiving the endoprosthesis is damaged, particularly in the area of the end of the bone (bone head). The reason for this is, in particular, defects in the (spongy and/or cortical) bone substance or surface, e.g., due to illness or injury, but increasingly also due to the explantation of an older prosthesis. In order to still create a sufficient base in the bone to anchor the endoprosthesis, augments are typically used to fill in the missing bone substance. It has proven useful to arrange them sunk into the end of the bone so that they are surrounded like a quiver by the cortical edge of the end of the bone.

Disk-like augments for use with the tibial component of a knee joint endoprosthesis are known, for example, from EP 1 360 950 B1. Two half-sided augments are provided below the tibial plate, which are arranged to the left and right of an anchoring keel. The augments abut flush against the bottom of the tibial plate and are, in the assembled state, secured there with a fastening screw. It has been shown that in this way the augments are held in a defined manner in relation to the endoprosthesis, but not in relation to the bone head that they actually are supposed to support or whose bone defect they are supposed to fill. A further difficulty can arise after long-term use because the augment grows into the bone over time and the endoprosthesis with the augment is then difficult or almost impossible to remove, for example as part of revision surgery. There is a risk of further damage to the already sensitive bone head.

The invention is based on the object of creating an improved augment with which the aforementioned disadvantages can be avoided or reduced.

The solution according to the invention is found in the features of the independent claim. Advantageous refinements are the subject of the dependent claims.

In a disk-shaped augment for filling bone defects, in particular at the end of long tubular bones such as the tibia, with a proximal side, a distal side, an outer sheath on lateral sides, and an inner wall for a through-opening running from the proximal side to the distal side for an anchoring keel of an endoprosthesis arranged on the proximal side, wherein the augment has a generally C-shaped shape with two legs flanking the through-opening, according to the invention it is provided that a connecting piece between the legs is formed in an articulated manner and interacts resiliently with the legs, so that when the legs are compressed, an outwardly directed restoring force is generated, and the outer sheath is designed as a porous structure which promotes bony ingrowth, and a cover plate is provided on the proximal side, which has a ribbed structure as an interlocking for cement bonding.

Some terms used are explained below:

The compression of the legs is understood to mean a movement of the legs towards each other, narrowing a free space between them.

A ribbed structure is preferably understood to mean a coarsely ribbed structure, with a distance between individual profiles of the ribbing of at least one millimeter. It has been shown that such a coarse ribbing creates a reliable and long-term stable positive fastening to bone cement.

For the ribbed structure, sharp-edged is understood to mean that protruding areas of the ribbing are designed to be without rounded edges.

An anchoring keel of an endoprosthesis is understood to mean a structure that is provided for anchoring the endoprosthesis in a bone, in particular a medullary canal of the bone, and comprises a shaft and wing-like extensions arranged laterally thereon.

For contour-like objects, “undersized” is understood to mean that the outer contour of one object runs within the outer contour of the other object, preferably reduced in dimensions by a certain absolute or relative amount.

The porous structure preferably has a porosity in the range of 60% to 90% and/or an average pore size of 0.1 mm to 1.5 mm, in particular 0.4 mm to 1.0 mm. The thickness of the porous structure is preferably between 0.6 mm and 2.5 mm, the thickness being dimensioned such that at least one layer of pores located in the depth of the material (i.e. not lying on the surface or in section) is provided.

The resilient design of the disk-shaped augment allows it to be inserted and sunk into a cavity surrounded by the cortex on the bone head. In doing so, the disk-shaped augment abuts against the inside of the cortex with its outer surface, and the compression of the legs during insertion presses the porous structure on the outer sheath against the inside of the cortex. This creates an intimate connection between the porous structure on the one hand and the cortex on the other, which creates a favorable primary fixation and subsequently the best conditions for the ingrowth of bone material and thus for a correspondingly long-term stable fastening. The cover plate separates bone cement applied to the top of the augment from the inner and lower areas of the augment, so that no disruptive fixation by bone cement occurs there and the ingrowth of bone material is not disrupted by penetrating cement. This in turn makes it possible to create the connection between the augment and the proximal endoprosthesis above it, for example the tibial plate of a knee joint endoprosthesis, by means of a cement bed. Thanks to the ribbing on the proximal side of the augment according to the invention, this results in a good hold of the cement bed and thus also of the proximally arranged endoprosthesis. The cover plate thus also creates the prerequisite for creating a cutting layer by means of the applied cement bed, which can be cut relatively easily using a saw in the event of a planned explantation, whereby the endoprosthesis is freed from the augment and can therefore be easily removed without tearing out the augment, which may have grown strongly into the bone head.

The invention is able to combine an independent fastening of the augment by means of the porous outer sheath, which is pressed against the cortex on the bone head by means of a pre-tensioning force applied by compression and thus creates optimal conditions for bone ingrowth, with a cover plate which enables the application of a cement layer which is fixed to the augment thanks to the ribbing and thus fastens the endoprosthesis lying above it, and additionally acts as a cutting layer at the end of the service life for easy separation of the endoprosthesis from the augment grown into the bone, without damaging the bone. The invention thus combines seemingly contradictory advantages in terms of fastening security on the one hand and damage-free explantation on the other. Revision operations with replacement of the endoprosthesis thus lead to a reduction in the risk for the patient.

The distal side of the disk-shaped augment is preferably designed as a porous structure. This creates favorable conditions for bone ingrowth and thus good fastening of the augment on the large contact surface with which the distal side abuts flat against the bone. The edges are advantageously designed to be solid. Edges are understood to mean the transitions between side surfaces, in particular from the distal side to lateral sides. This results in a reinforcement of the edges, which prevents the porous structure from breaking off.

The porous structure is advantageously designed in such a way that it comprises connected pores in the depth of the material. The porosity therefore goes beyond superficial porosity and extends into the depths of the material. In this way, cavities are created that are connected to one another, which creates particularly favorable conditions for the ingrowth of bone material. The pores are advantageously dimensioned so that they have a width of about 0.4 mm to 1.0 mm. This results in particularly favorable ingrowth characteristic. Expediently, the porous structure comprises at least one and up to three layers of pores in the depth, which typically means a preferred thickness of the porous structure of about 0.6 mm to 2.5 mm.

It is expediently provided that the porous structure has porous areas that are framed by a solid edge. This, on the one hand, achieves a clear limitation and, on the other hand, prevents undesirable breaking out of mechanically more sensitive porous material from the porous structure, in particular in the edge area.

It is possible for the legs to be formed from a porous structure, with the proximal side of course still being provided with a cover plate, as stated above. On the one hand, this results in a light-weight structure that, on the other hand, is almost completely open for the ingrowth of bone material. However, it is preferred that the legs have a solid (non-porous) core, on the outside of which pockets are formed in which the porous structure is arranged. This allows defined zones to be created where the porous structure is present and where the bone material is supposed to grow in. In addition, the legs or the augment become(s) mechanically more robust and sturdy, and unnecessarily deep ingrowth of the bone material is prevented. The latter only provides minor advantages in terms of secure fastening as the depth increases, but such particularly deep ingrowth of bone material usually means considerable difficulty when explanting the augment. The arrangement of the pockets can limit the ingrowth depth, whereby, as already mentioned above, the porous structure comprises at least one layer of pores connected in the depth of the material.

The inner wall on the legs is advantageously designed to be solid. Ingrowth of bone material at this difficult-to-access location is typically not desired, especially since the inner wall typically only has contact with the anchoring keel and other fastening elements, such as, for example, screws. This also applies to lateral surfaces other than the outer sheath, e.g. this also applies to inner walls between which slots or gaps are formed.

In a particularly preferred embodiment, which may deserve independent protection, the legs are designed in a skeletal construction with several adjacent disk segments separated by slots, which are arranged on a frame via flexible webs. The disk-shaped augment is therefore not designed as a unitary disk (with a through-opening), but is divided into several adjacent disk segments. They typically have the same thickness. The disk segments are fastened to the frame via flexible webs. This enables compression of the legs without causing tension between the disk segments. The skeletal construction enables particularly favorable compression and resilient characteristics, which simplifies the insertion of the augment into a recess created in the bone head and ensures increased fastening security there. A sufficient sealing effect against bone is provided, so that a continuous sealing plate is not required in this embodiment; it is sufficient if each of the disk segments forms its own cover plate. Another advantage of the skeletal construction is that augments of different sizes can be formed by adding or removing one or more disk segments.

Preferably, the slots are so narrow that they act as a gap seal for bone cement. Adequate sealing against bone cement is thus achieved in a simple manner with little effort. Preferably, the slots have preferably a maximum width of 1 mm, more preferably a maximum width of 0.7 mm. Depending on the height of the disk segments larger widths may suffice or smaller widths may be required to obtain an adequate sealing gap.

The lateral surfaces of the disk segments (usually the lateral outer sides or the side surfaces forming the slots) are advantageously designed to be solid, i.e. nonporous. This creates rigidity. Furthermore, in doing so, the risk of undesired penetration of bone cement into or through the slots is effectively minimized. The disk segments are preferably designed to be solid, if necessary, with pockets for porous structure on the outer sheath and/or on the distal side.

Expediently, the frame is designed as an outer edge which encloses at least half of the legs, on the outside of which the outer sheath is arranged. The frame borders the entirety of the disk segments, which are thus located on the inside of the frame. At the same time, the frame can thus form the outer sheath with the porous structure. The stress load resulting from the compression is distributed over the frame, which leads to an even load and thus to a higher load capacity.

Preferably, at least one fastening hole is provided in the legs for receiving a fastening screw. This allows additional fastening of the augment to the bone, which can make a valuable contribution to the primary fastening in particular. The fastening hole advantageously has a solid perforated sheath. Such a lining prevents ingrowing bone material from creating a connection between the screw thread and the porous structure of the augment, which would make it more difficult to loosen the screw.

For skeletal construction, it is expedient if the fastening hole is arranged in one of the disk segments, wherein this disk segment is not arranged on the frame by means of a narrow web, but rather by means of a wider bridge, which has a width of at least 2 times the width of the webs. This achieves two advantages: on the one hand, an efficient fastening of the disk segment, which is additionally loaded by the fastening screw, is achieved. On the other hand, the wider bridge makes it possible to move the fastening hole closer to the edge and thus closer to the frame, which is a significant advantage, especially with small augment sizes, due to the improved use of space.

The through-opening is typically an elongated receiving space for an anchoring keel of the endoprosthesis. The through-opening can expediently be open towards one side. This is particularly advantageous if the augment is designed as a half-sided augment. The term half-sided refers in this case to the dimensions of the endoprosthesis, whose anchoring keel is guided through the through-opening of the augment. The endoprosthesis often only requires relining on one side, for example if a bone defect is limited to one side of the bone head and the augment is therefore only needed there. It should be understood that two half-sided augments can also be provided in order to provide relining on both sides.

The through-opening is typically rather small in relation to the dimensions of the augment; typically, it occupies less than half, often less than one third, of the volume of the augment. This has the advantage that still sufficient supporting material for the legs or the disk segments remains to ensure sufficient relining. The through-opening is usually dimensioned so that it can receive the anchoring keel or, if necessary, its wing-like extensions, with little clearance. The little clearance is expediently chosen such that there is a sufficient sealing effect against bone cement.

The outer sheath is advantageously not arranged at a 90° angle to the proximal side or to the distal side. It has proven useful if the outer sheath is conically inclined, preferably tapering towards the distal side at an angle of 5 to 10°. Due to this conicity, the augment can be inserted or removed more easily by pressing than would be the case with a vertical outer sheath.

Outwardly directed spikes are expediently arranged on the outer sheath. When implanted, they press into the surrounding cortex of the bone head, in which the augment is received in a recess. This achieves a more reliable fastening right from the start, i.e. improved primary fixation. When the legs of the augment are pressed together (compression) for explantation, the spikes then automatically move back out of the cortex, thereby enabling explantation.

The ribbed structure is advantageously designed to have grooves. Expediently, continuous grooves are provided, which are preferably oriented in a direction substantially transverse to the extent of the through-opening. The grooving achieves particularly effective interlocking with an applied cement bed. The grooves preferably have such spacing and height that is tailored to the flow and setting characteristic as well as the grain size of the cement bed. Groove spacings in the range of 2 to 10 mm and/or a groove depth of 0.5 to 2 mm have proven useful, with profiles delimiting the grooves being about 0.5 to 3 mm wide.

The ribbed structure can be designed to be sharp-edged. However, other alternative profile shapes are also conceivable, alternatively triangular shapes or profile shapes with rounded corners.

Expediently, the ribbed structure is produced using a wire erosion process. In doing so almost any profile shape, including sharp-edged ones, can be formed efficiently.

It is particularly expedient if the augment is produced uniformly using an additive process, in particular 3D printing. In doing so even complex shapes in different sizes can be produced without or only with a minimum of complex post-processing. A combination with wire erosion is particularly expedient for creating defined, sharply delimited profiles, as mentioned above.

The cover plate can be designed to be cement-tight. Cement-tight is understood to mean that no bone cement passes through the cover plate. For this purpose, the cover plate can be designed to be continuous or provided with, in particular, slot-like openings which act as seals, in particular gap seals, against the entry of bone cement.

Advantageously, it is provided that the porous structure is provided with a coating that promotes bone ingrowth, in particular comprising calcium phosphate, and/or is provided with a biocidal coating, in particular comprising silver, on non-porous areas. This ensures that ingrowth of bone material is favored at desired places and that such ingrowth does not happen at other places where it is not desired, especially for the purpose of easier explantation.

The invention further includes an arrangement consisting of an endoprosthesis, in particular a knee joint endoprosthesis, and the disk-shaped augment. An anchoring keel of the endoprosthesis is received in the through-opening, particularly in the implanted state. The disk-shaped augment is expediently configured in its outer contour similar to the endoprosthesis, in particular a tibial plate. However, in comparison, said augment is undersized, i.e. it has somewhat smaller dimensions (particularly in the range of 2 to 6 mm along the outer sheath). As a result, it can support the endoprosthesis, especially the tibial plate, but nevertheless received in a recess bordered by the cortex at the bone head.

The disk-shaped augment is advantageously designed to support a left or right half of the endoprosthesis on one side. As a result, effective support can be achieved by the augment if a bone defect is only present on one side of the bone head, thus avoiding the unnecessary removal of bone material on the other, healthy side.

The through-opening for receiving the anchoring keel and its wing-like extensions is expediently dimensioned with a defined free space of preferably 1 to 3 mm. This free space creates a clearance between the anchoring keel with its wing-like extensions and the legs of the augment, which enables tolerance compensation and unnecessary direct contact is avoided. In practice it is a considerable relief, as undesirable jamming of the augment and anchoring keel with its wing-like extensions can be avoided.

The invention is explained below with reference to the accompanying drawing using advantageous exemplary embodiments. In the drawing:

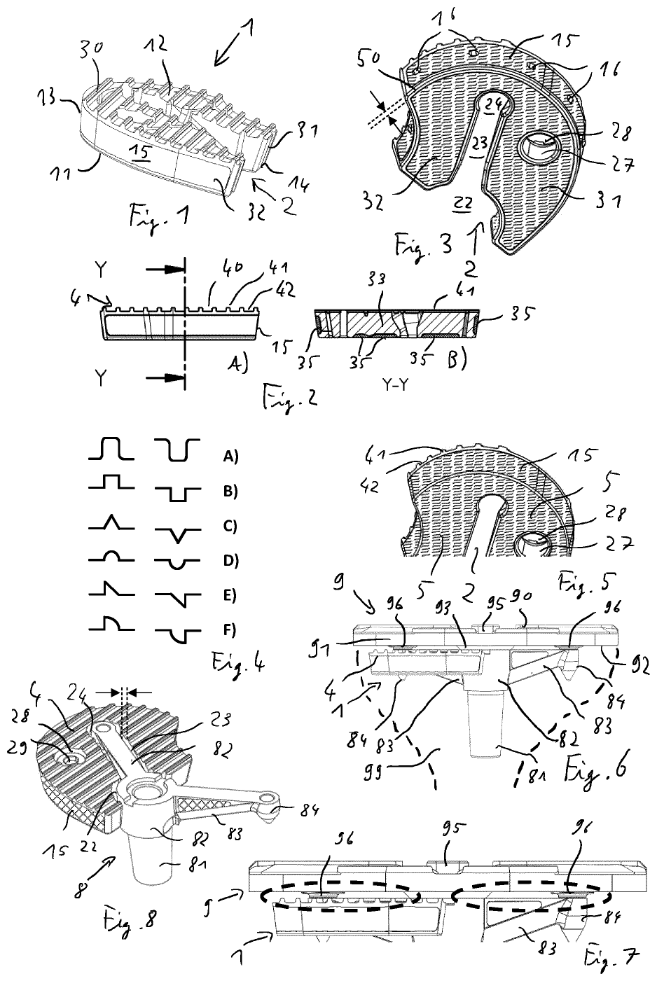

FIG. 1 shows a perspective view of a first exemplary embodiment for a disk-shaped augment;

FIGS. 2A, B show a rear view and a medial sectional view of the augment according to FIG. 1;

FIG. 3 shows a perspective distal view illustrating an outer sheath and a distal side of the augment;

FIG. 4 shows different profile shapes for ribbing;

FIG. 5 shows a detailed view illustrating legs with porous design according to a variant of the augment;

FIG. 6 shows a frontal view of the augment together with a tibial plate of a knee joint endoprosthesis;

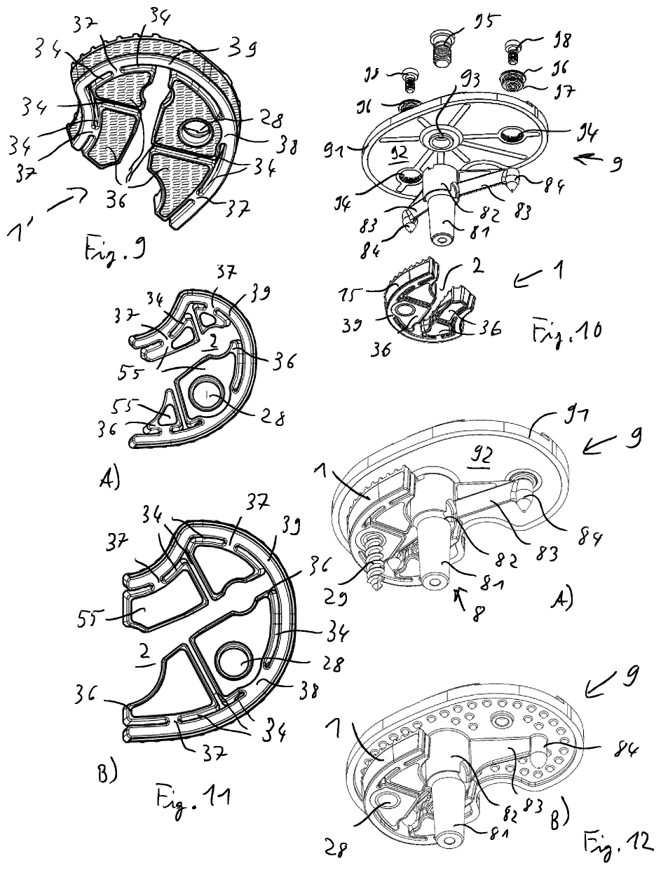

FIG. 7 shows an enlarged detailed view for arranging the augment on the knee joint endoprosthesis;

FIG. 8 shows a detailed view of the augment with inserted anchoring keel of the knee joint endoprosthesis;

FIG. 9 shows a perspective distal oblique view of a second exemplary embodiment of the augment;

FIG. 10 shows an exploded view of the augment according to FIG. 9 with the knee joint endoprosthesis;

FIGS. 11A, B show top views of a distal side of the augment in two different sizes; and

FIGS. 12A, B show the augment with different tibial plates of a modular knee joint endoprosthesis.

The invention is explained below using an example of an augment for a knee joint endoprosthesis, specifically for an augment arranged on the upper (proximal) bone head of the tibia. The augment must be distinguished from the prosthesis, i.e. the augment is not an element of the actual prosthesis. The augment strengthens the bone and thus increases or improves its ability to accommodate the prosthesis. Augments according to the invention can of course also be provided on other bones. Augment 1 preferably consists of a titanium alloy, and can expediently be produced using an additive process (3D printing). However, it can also consist of other biocompatible material, for example metallic material such as, for example, cobalt-chromium-molybdenum (CoCrMo) or plastic material, such as, for example, polyether ether ketone (PEEK).

Augment 1 is provided to be arranged below tibial component 9 of a knee joint endoprosthesis, as shown in FIG. 6. The knee joint endoprosthesis comprises a femoral component (not shown) to be arranged on the femoral bone (not shown) and tibial component 9 to be arranged at the proximal end of a tibial bone 99. Said tibial component 9 comprises a tibial plate 91 which is arranged laterally extended on a resected bone head of the tibia. On the proximal side (top side) of tibial plate 91, a receptacle 90 is provided, in which a bearing piece (not shown) of the knee joint endoprosthesis is to be arranged. The opposite, distal side 92 of tibial plate 91 is designed for abutment against the surface of the bone head of tibia 99. Depending on whether the tibial component is provided for cemented or cement-free implantation, tibial plate 91 is optionally provided on its distal side 92 with a porous structure for the ingrowth of bone material in order to enable optimal fastening with cement-free implantation.

To fasten tibial plate 91, an anchoring keel 8 is provided, which projects distally. It has a shaft piece 82 and a distally adjoining cone piece 81. Cone piece 81 is designed to, if necessary, to receive a plug-in shaft protruding into the medullary canal of tibia. Laterally, shaft piece 82 is adjoined by a wing-like extension 83 on both the left and right sides, at the free end of which a clamping sleeve 84 is arranged. Wing-like extensions 83 act as support arms for tibial plate 91. Tibial plate 91 is thus connected to anchoring keel 8 at three points, that is to say centrally on shaft piece 82 and on each of the left and right sides with clamping sleeves 84 arranged at the free ends of wing-like extensions 83. For this purpose, two screw holes 94 are provided on tibial plate 91 to the left and to the right, which are aligned with respective clamping sleeve 84.

For this purpose, wing-like extensions 83 are not abutting flush against bottom 92, but are held at a defined distance. For this purpose, a central support collar 93 is provided on bottom 92, against which shaft piece 82 abuts. Spacer inserts 96 with an external thread 97 are screwed into screw holes 94 (see FIG. 10), which create a defined distance from clamping sleeves 84. In this way, together with the central support collar 93, a defined distance is set between bottom 92 of tibial plate 91 and wing-like extensions 83 of anchoring keel 8. This is shown in FIG. 6, and the distance set in a defined manner is highlighted in the detailed representation according to FIG. 7 by the ellipses shown with a dotted line.

FIG. 1 shows a perspective view of a first exemplary embodiment of an augment 1 according to the present invention. Augment 1 is shaped like a thick disk and has two legs 31, 32 which are coupled resiliently in an articulated manner via a connecting piece 30 at the common end of legs 31, 32. A through-opening 2 is arranged between legs 31, 32, which is designed to receive an anchoring keel 8 of tibial component 9.

A rear view of augment 1 and a medial sectional view are shown in FIGS. 2A and B. Here, the rear view refers to the orientation of augment 1 in the inserted state, that is to say, using anatomical terms, this is a posterior view. On top, i.e. on the proximal side, a cement-tight cover plate 4 is formed on augment 1. Said cover plate is provided with a ribbed structure 40 on its top. Ribbed structure 40 thus serves to interlock a cement bed (not shown), which fills the space between tibial plate 91 and thus provides additional fastening. Ribbing 40 can have grooves 42, the individual grooves being separated from one another by raised profiles. The grooves are oriented in a direction from front to back, i.e. from anterior to posterior in relation to the implanted state. The depth of grooves 40 is, for example, 1 mm, the width of the grooves about 6 mm and the width of raised profiles 41 about 1.5 mm. In the simplest case, profiles 41 can have a rectangular cross-sectional shape, as shown in FIG. 5A) or 5B), with rounded or sharp corners, each as a positive or negative profile. Alternatively, non-rectangular shapes for the profile are also possible, for example in the form of a symmetrical triangle or an asymmetrical, sawtooth-like triangle, as shown in FIG. 5C) or 5E). Furthermore, rounded profiles can also be provided, for example in the manner of a semicircle as shown in FIG. 5D) or asymmetrically in the manner of a quarter circle as shown in FIG. 5F). The person skilled in the art can make a selection depending on the requirements for the interlocking with the bone cement formed by profiles 41.

Furthermore, in FIGS. 2A, B it is apparent from the respective lateral sides of augment 1 that they taper conically towards the distal side (in the figure downwards). In the exemplary embodiment shown, the angle on each side is about 7°. Other angles can also be provided. The conical configuration not only simplifies insertion during implantation, but above all it also simplifies removal of augment 1 upwards in the context of an explantation.

Reference is now made to FIG. 2B) and FIG. 3. As visualized by a dashed line representation in FIG. 3, distal bottom 11 of augment 1 and an outer sheath 15 on lateral side 13 are each provided with a porous structure 5. In the embodiment shown, the porous structure is not continuous, but legs 31, 32 have a solid core 33. Pockets 35 are provided on the corresponding surfaces of legs 31, 32, in which porous structure 5 is arranged. Pockets 35 have a depth of about 0.8 to 1.5 mm. In FIG. 2B, such pockets are provided and shown on distal (side 11 and laterally on outer sheath 15. In order to prevent undesirable breakage of porous structure 5, in particular at the edges of augment 1, the edges between adjacent porous structures 5 at the transition from the distal side to the lateral side are provided with solid, i.e. non-porous, edges 50 as a frame. Thus, reinforcement is achieved and porous structure 5 is prevented from breaking away. The width of these edges 50 is visualized by the two opposing arrows in FIG. 2 and is preferably about 1 mm to 2 mm.

Furthermore, spikes 16 pointing radially outwards can be arranged on outer sheath 15. After the implantation of augment 1, these spikes drill into the surrounding cortex of the tibial head and thus additionally secure augment 1 in its position. Furthermore, a fastening hole 38 is provided on leg 31. It is dimensioned such that a cancellous bone screw 29 can be inserted and tightened in this opening for additional fastening. For additional rigidity, the fastening hole 28 is provided with a solid perforated sheath 27 as an inner lining.

Furthermore, as can be clearly seen from FIG. 3, through-opening 2 is divided into several areas. The largest area is taken up by a receiving area 22 for the shaft piece 82 of the anchoring part 8 at the edge of the through-opening, to which an elongated area 23 of the through-opening extends, which is designed to receive the wing-like extension 83. At the far end thereof, a circular receiving area 24 is provided for the clamping sleeves 84 at the free end of the respective wing-like extension 83. As can be seen in particular by comparison with FIG. 8, through-opening 2 with its areas 22, 23 and 24 is matched to the anchoring part 8 in terms of dimensions. This results in the anchoring part 8 being received with clearance, but said clearance is relatively small and amounts to about 1.5 mm in the example shown in FIG. 8, with this clearance distance being visualized by the two opposing arrows in FIG. 8. This small clearance creates the effect of a gap seal to anchoring keel 8.

FIG. 5 shows a variant of the augment shown in FIG. 3. Unlike in FIG. 3, there is no solid core 33 for legs 31, 32, but these are formed continuously from porous structure 5, except for cover plate 4 on proximal side 12. This design is very light weight and allows good ingrowth of bone material all around. Widened edges 30 for protecting the porous structure 5 are no longer present in the critical edge area, so that there is an increased risk of components breaking out of porous structure 5.

A second exemplary embodiment of the augment is shown as a perspective distal oblique view in FIG. 9. Similar elements as in the first exemplary embodiment have the same reference numerals, with different components having different reference numerals. In this embodiment, the disk-shaped augment 1′ is designed in a skeletal construction. For this purpose, it has a frame 39 that runs around at least three-quarters of the circumference of augment 1′. In the exemplary embodiment shown, a plurality of disk segments 36 are arranged on the inside of this frame, with through-opening 2 being formed between disk segments 36. Through-opening 2 with its various areas 22, 23 and 24 is configured as in the first exemplary embodiment, to which reference is hereby made to avoid unnecessary repetition.

Disk segments 36 are each resiliently arranged on the inside of frame 39 via a web 37. As a result, narrow slots 34 are formed between disk segments 36 as well as between disk segments 36 and the inside wall of frame 39, which provide space for relative movement of the disk segments during compression/expansion. The width of slots 34 is dimensioned such that, taking into account the depth of slots 34 determined by the thickness of augment 1′, a gap seal is formed that is effective against the ingress of bone cement. In this way, the flexibility of disk segments 36, which are flexibly arranged over one of webs 37 in each case, can be combined with a construction that prevents the ingress of bone cement in a simple and robust manner. These disk segments 36 can be used to finely adapt to the space required by through-opening 2, which in turn is largely determined by the nature of the anchoring keel 8 of the prosthesis to be implanted.

Furthermore, disk segments 36 make it possible to easily provide variants of the augment in larger or smaller sizes. For example, by omitting one of disk segments 96, a smaller size of augment 1 can be readily formed, as shown in FIG. 11A), or a larger size can be formed by adding a disk segment, as shown in FIG. 11B). In this way, synergy effects arise when augments according to the invention are provided in an augment set with different sizes.

Porous structure 5 can be provided with a biocompatible coating 55, for example made of calcium phosphate, to further promote bone ingrowth. This applies to porous structure 5 of all embodiments.

Fastening hole 28 can be arranged in one of shell segments 36. As in the first embodiment described above, fastening hole 28 is expediently provided with a perforated sheath 27 as an inner lining. Furthermore, the invention makes it possible to bring fastening hole 28 closer to the edge of the disk segment, preferably close to surrounding frame 39. To make this possible, the web is expediently widened significantly at this point to form a bridge 38, which offers additional space in order to arrange fastening hole 28 as close as possible to frame 39. In this way, a uniform arrangement of fastening hole 28 and cancellous bone screw 29 arranged thereon is achieved across different prosthesis sizes.

An exploded view with respect to the second exemplary embodiment is shown in FIG. 10. The main components are apparent to be augment 1 and tibial plate 91 with its anchoring keel 8. As already described above, installation or assembly is carried out by fastening anchoring part 8 with a shaft piece 82 to the bottom of distal side 11 of tibial plate 91 by means of central screw 95. In addition, spacer inserts 96 made of titanium material with an external thread 97 are screwed into screw holes 94 and thus determine a defined distance between wing-like arms 83 and distal side 92 of tibial plate 91. Furthermore, for additional fastening and support, fastening screws 98 are provided, which are passed through respective spacer insert 96 and engage in an internal thread of respective clamping sleeve 84 and thus clamp it against spacer insert 96. In this way, a defined distance is set between wing-like arms 83 and distal side 92 of tibial plate 91.

This distance creates space for a saw slot into which a saw can be inserted during an explantation. This saw slot is shown in FIG. 7 (see, in particular, the area marked by the dashed ellipse). For explantation, fastening screws 98 are unscrewed and spacer inserts 96 are also removed. The saw slot is now accessible from the side up to shaft piece 82 in the middle of tibial plate 91. The bottom of tibial plate 91 can thus be cut out by means of a saw (not shown). Finally, central screw 95 is removed and tibial plate 91 can now be removed. In this way, augment 1 with a cement layer possibly arranged thereon, is accessible, which cement layer can then also be removed if necessary. If desired, augment 1 can be explanted upwards by pressing legs 31, 32 together.

Augment 1 according to the invention is particularly suitable for use in a modular knee joint endoprosthesis. Examples of this are shown in FIGS. 12A, B. The respective augment is arranged on one side of anchoring keel 8, wherein different tibial plates 91 can be provided in each case. In FIG. 12A, a tibial plate 91 with a porous distal side 92 is provided, while in FIG. 12B a differently configured tibial plate 91′ is provided. Furthermore, FIG. 12A shows that augment 1 can be additionally fastened by means of a cancellous bone screw 29. It is understood that such a cancellous bone screw, which is received in fastening hole 28, can also be provided in the other embodiments shown.

1. A disk-shaped augment for filling bone defects having a proximal side, a distal side, an outer sheath on lateral sides, and an inner wall for a through-opening running from the proximal side to the distal side for an anchoring keel of an endoprosthesis arranged on the proximal side, wherein the augment is C-shaped with two legs flanking the through-opening, characterized in that a connecting piece between the legs is formed in an articulated manner and interacts resiliently with the legs so that when the legs are compressed an outwardly directed restoring force is generated, and
the outer sheath is designed as a porous structure which promotes bone ingrowth, and a cover plate is provided on the proximal side, which cover plate has a ribbed structure as an interlocking for cement bonding.

2. The disk-shaped augment according to claim 1, wherein the distal side is designed as a porous structure, wherein edges solid.

3. The disk-shaped augment according to claim 1, wherein the porous structure comprises pores connected to one another in a depth of the porous structure, wherein the pores have a width of 0.4 to 1.0 mm.

4. The disk-shaped augment according to claim 1, wherein the porous structure has porous areas that are framed by a solid edge.

5. The disk-shaped augment of claim 1, wherein the legs have a solid core, on an outside of which pockets are formed, in which the porous structure is arranged.

6. The disk-shaped augment according to claim 1, wherein the legs are formed from a porous structure.

7. The disk-shaped augment according to claim 1, wherein the inner walls on the legs are solid.

8. The disk-shaped augment according to claim 1, wherein the legs comprise a skeletal construction with multiple adjacent disk segments separated by slots, and arranged on a frame via flexible webs, wherein the frame is an outer edge which encloses at least half of the legs, and on an outside of which the outer sheath is arranged.

9. The disk-shaped augment according to claim 7, wherein the slots are dimensioned so narrowly that they act as a gap seal for bone cement.

10. The disk-shaped augment according to claim 1, wherein at least one fastening hole for receiving a fastening screw is provided in the legs, wherein the fastening hole has a solid perforated sheath.

11. The disk-shaped augment according to claim 10, wherein the fastening hole is arranged in a disk segment, wherein said disk segment is arranged on a frame by means of a bridge, wherein the bridge is at least twice as wide as a flexible web that connects the disk segment to the frame.

12. The disk-shaped augment according to claim 1, wherein the through-opening is an elongated receiving space for an anchoring keel and/or the through-opening is open on one side, and/or the through-opening occupies less than half of a volume of the augment.

13. The disk-shaped augment according to claim 1, wherein the outer sheath is conically inclined, tapering towards the distal side at an angle of 5 to 10°.

14. The disk-shaped augment according to claim 1, further comprising radially outwardly directed spikes provided on the outer sheath.

15. The disk-shaped augment according to claim 1, wherein the ribbed structure has continuous grooves.

16. The disk-shaped augment according to claim 1, wherein the ribbed structure is produced using a wire erosion process and/or the augment is produced uniformly using an additive process.

17. The disk-shaped augment according to claim 1, wherein the cover plate is cement-tight.

18. The disk-shaped augment according to claim 1, wherein the porous structure is provided with a bone ingrowth-promoting coating comprising calcium phosphate, and/or is provided on non-porous areas with a biocidal coating.

19. An arrangement of an endoprosthesis and the disk-shaped augment according claim 1, wherein an anchoring keel of the endoprosthesis is received in the through-opening.

20. The arrangement according to claim 19, wherein the disk-shaped augment is similar in size to an outer contour but undersized in relation to the endoprosthesis.

21. The arrangement according to claim 19, wherein the disk-shaped augment is configured to support a left half or a right half of the endoprosthesis.

22. The arrangement according to claim 19, wherein the through-opening is dimensioned for receiving the anchoring keel and its wing-like extensions with a defined free space of 1 to 3 mm.
